(19) United States
(12) Patent Application Publication
(10) Pub. No.: US 2018/0344282 A1
(43) Pub. Date: Dec. 6, 2018

(54) DIAPHRAGM AND STETHOSCOPE HEAD ASSEMBLY THEREOF

(71) Applicant: VITALCHAINS CORPORATION, Menlo Park, CA (US)

(72) Inventors: TZU-CHIH LIN, Hsinchu (TW); YU CHENG HUANG, Hsinchu (TW)

(21) Appl. No.: 15/995,153

(22) Filed: Jun. 1, 2018

Related U.S. Application Data
(60) Provisional application No. 62/514,037, filed on Jun. 2, 2017.
(60) Provisional application No. 62/584,215, filed on Nov. 10, 2017.

(30) Foreign Application Priority Data
Sep. 6, 2017 (TW) 106130520

(51) Int. Cl. A61B 7/02

(52) U.S. Cl. CPC A61B 7/02; G10K 11/18

(57) ABSTRACT

The present disclosure provides a stethoscope head assembly. The stethoscope head assembly comprises a diaphragm and a main body. The diaphragm comprises a membrane portion, and a sealing portion extending from a periphery of the membrane portion and comprising a ring-shaped raised platform and a connecting portion extending downwardly from a periphery of the ring-shaped raised platform. The main-body is detachably coupled to the diaphragm and comprises a sound gathering member. The connecting portion and the ring-shaped raised platform of the diaphragm cooperatively form a ring-shaped receiving slot for receiving the sound gathering member of the main body.

### CROSS-REFERENCE TO RELATED APPLICATION

The present disclosure claims priority to U.S. provisional patent application No. 62/514,037 filed on Jun. 2, 2017, U.S. provisional patent application No. 62/584,215 filed on Nov. 10, 2017, and Taiwanese invention patent application No. 106130520 filed on Sep. 6, 2017, the entirety of which are incorporated herein by reference.

### FIELD OF THE INVENTION

The present disclosure relates to a stethoscope head assembly and a diaphragm for the stethoscope head assembly.

### BACKGROUND OF THE INVENTION

A number of different stethoscope head assemblies are commercially available. Conventional stethoscope head assembly usually is consisting of a main body, a polycarbonate (PC) diaphragm, and a ring-shaped or an O-ring-shaped metal. The main body is coupled to the PC diaphragm by threading the O-ring-shaped metal with the main body.

A setback of the conventional stethoscope head assembly is the process of assembling the components of the stethoscope head assembly. The assembly process is time-consuming and difficult for multiple components to be accurately aligned with a particular area of the diaphragm. The assembly between the PC diaphragm and the O-ring are sometimes not sealed properly, causing a small conducting surface of the PC diaphragm, thus the sound gathering ability of the conventional stethoscope head assembly is poor.

Another setback of conventional stethoscope head assembly is frictional noises generated when being used to auscultate the human body. When the stethoscope head assembly is removed from a body of the patient, slide through the body, or changed to another side of the stethoscope head assembly, frictional noises often occur, and the volume of the frictional noises is correlated to the size of a frictional area. The frictional area between a patient and the diaphragm is a contact surface of the diaphragm.

Yet another setback of conventional stethoscope head assembly occurs in the replacement of diaphragms. After an examination by the stethoscope head assembly on the body is completed, conventional diaphragm of stethoscope of the stethoscope head assembly needs to be either replaced or disinfected by alcohol after being used to ensure hygiene and prevent infection. When replacing the diaphragm, poor sealing between the diaphragm and the main body often occurs, which may cause bad auscultating experience.

Additionally, because conventional PC diaphragm is made of a hard material, the process of forcing the diaphragm of the stethoscope head assembly onto the patient often causes discomfort.

Furthermore, considering the various diagnostic applications of the physiological sounds from the chest, it is desirable for the physician to switch between high and low frequencies when using the stethoscope head assembly. The physician usually switches sides of the stethoscope head assembly to detect high and low frequency sounds. However, the process of switching sides of the stethoscope head assembly makes it difficult to locate the precise area of the body of the patient.

### BRIEF SUMMARY OF THE INVENTION

In view of the above-described circumstances, the necessary objective of the present disclosure is to provide a stethoscope head assembly that has excellent sound gathering ability, low frictional noises and easy to switch between high and low frequencies.

An embodiment of the present disclosure provides a diaphragm for a stethoscope head assembly. The diaphragm comprises a membrane portion and a sealing portion. The sealing portion extends from a periphery of the membrane portion and comprises a ring-shaped raised platform and a connecting portion extending downwardly from a periphery of the ring shaped raised platform. The connecting portion and the ring-shaped raised platform cooperatively form a ring-shaped receiving slot.

In a preferred embodiment, the diaphragm is made of an elastic material with a Shore A hardness of 30 to 80.

In a preferred embodiment, the elastic material comprises silicon, thermoset rubber, thermoplastic rubber, or a combination thereof.

In a preferred embodiment, a thickness of the membrane portion is less than a thickness of the ring-shaped raised platform of the sealing portion.

In a preferred embodiment, the thickness of the membrane portion falls within a range of 0.05 mm to 1 mm.

In a preferred embodiment, a diameter of the membrane portion falls within a range of 30 mm to 50 mm.

In a preferred embodiment, the ring-shaped raised platform comprises a platform top surface, a platform bottom surface, a ring-shaped first side wall, and a ring-shaped second side wall. The ring-shaped first side wall protrudes inwardly from a periphery of the platform top surface. The ring-shaped second side wall protrudes outwardly from another periphery of the platform top surface. The connecting portion comprises a latching portion and a resisting portion coupled between the latching portion and the ring-shaped raised platform.

In a preferred embodiment, the ring-shaped first side wall of the ring-shaped raised platform and a top surface of the membrane portion form an angle, the angle falls with a range of 120 degrees to 180 degrees.

In a preferred embodiment, the sealing portion further comprises a first matching element arranged adjacent to the membrane portion at a bottom surface of the ring-shaped raised platform.

In a preferred embodiment, the first matching element is a recess.

In preferred embodiment, the first matching element is a protrusion.

In a preferred embodiment, the diaphragm further comprises a coating layer disposed on a bottom surface of the membrane portion and the ring-shaped raised platform.

In a preferred embodiment, the coating layer comprises silicon, soft touch paint, thermoplastic polyurethane, thermoplastic rubber, or a combination thereof.

In a preferred embodiment, a thickness of the coating layer of the diaphragm falls within a range of 0.04 mm to 0.1 mm.

In a preferred embodiment, the coating layer is disposed on the bottom surface of the membrane portion and the ring-shaped raised platform.

In a preferred embodiment, the diaphragm is manufactured by 3D printing.

Another embodiment of the present disclosure provides a stethoscope head assembly. The stethoscope head assembly comprises a diaphragm and a main body. The diaphragm comprises a membrane portion, and a sealing portion extending from a periphery of the membrane portion. The sealing portion comprises a ring-shaped raised platform, a first matching element arranged adjacent to the membrane portion at a bottom surface of the ring-shaped raised platform, and a connecting portion extending downwardly from a periphery of the ring-shaped raised platform. The main-body is coupled to the diaphragm and comprises a sound gathering member. The sound gathering member comprises a second matching element aligned to conform in shape with the first matching element of the ring-shaped platform at a top surface of the sound gathering member. The connecting portion and the ring-shaped raised platform of the diaphragm cooperatively form a ring-shaped receiving slot for receiving the sound gathering member of the main body.

In a preferred embodiment of the present disclosure, the stethoscope head assembly further comprises a relaxed state where the first matching element is not in contact with the second matching element, and a compressed state where the first matching element is in contact with the second matching element and buts against the second matching element. The stethoscope head assembly is transformed from the relaxed state to the compressed state by an external pressure.

In a preferred embodiment, a shape of the second matching element substantially corresponds to a shape of the first matching element.

In a preferred embodiment, the second matching element is a protrusion.

In a preferred embodiment, the second matching element is a recess.

In a preferred embodiment, an outer diameter of the ring-shaped receiving slot is substantially equal to an outer diameter of the sound gathering member.

In a preferred embodiment, the sound gathering member further comprises an engaging portion. The engaging portion is arranged on a periphery of the sound gathering member, and wherein a thickness of the engaging portion of the sound gathering member is substantially equal to a height of the ring-shaped receiving slot.

Another embodiment of the present disclosure provides a disposable capsule for containing a diaphragm for a stethoscope head assembly. The disposable capsule comprises a blister for disposing the diaphragm and a sealing film configured to seal the blister.

Another embodiment of the present disclosure provides a package for containing a plurality of disposable capsules. Each of the disposable capsules contains a diaphragm for a stethoscope head assembly. The package comprises a tearing portion arranged on a surface of the package and configured to form an opening after being torn off, and a plurality of the disposable capsules disposed in the package.

### BRIEF DESCRIPTION OF THE DRAWINGS

The accompanying drawings illustrate one or more embodiments of the present disclosure and, together with the written description, explain the principles of the present disclosure. Wherever possible, the same reference numbers are used throughout the drawings referring to the same or like elements of an embodiment.

FIG. 1 is a marked-up schematic view of a stethoscope head assembly in accordance with an embodiment of the present disclosure.

FIG. 2 is an exploded view of a stethoscope head assembly in accordance with an embodiment of the present disclosure.

FIG. 3 is a marked-up exploded cross-sectional view of the stethoscope head assembly in accordance with an embodiment of the present disclosure.

FIG. 4 is a marked-up cross-sectional view of the stethoscope head assembly along the line IV of FIG. 1, in accordance with an embodiment of the present disclosure.

FIG. 5 is another cross-sectional view of the stethoscope head assembly along the line IV of FIG. 1, in accordance with an embodiment of the present disclosure.

FIG. 6 is an exploded cross-sectional view of the stethoscope head assembly in accordance with an embodiment of the present disclosure.

FIG. 7 is another exploded cross-sectional view of the stethoscope head assembly in accordance with an embodiment of the present disclosure.

FIG. 8 are results of the decibel values gathered in different frequencies by stethoscope head assemblies using different diaphragms, in accordance with embodiments of the present disclosure.

FIG. 9 are steps of a manufacturing method for the diaphragm in accordance with an embodiment of the present disclosure.

FIG. 10 is a schematic view of a disposable capsule containing a diaphragm in accordance with an embodiment of the present disclosure.

FIG. 11 is a schematic view of a disposable capsule with a sealing film in accordance with an embodiment of the present disclosure.

FIG. 12 are steps of a method for replacing the diaphragm with the disposable capsule, in accordance with embodiments of the present disclosure.

FIG. 13 is a schematic view of a plurality of packages containing a plurality of the disposable capsules in accordance with an embodiment of the present disclosure.

In accordance with common practice, the various described features are not drawn to scale and are drawn to emphasize features relevant to the present disclosure. Like reference characters denote like elements throughout the figures and text.

### DETAILED DESCRIPTION OF THE PREFERRED EMBODIMENTS

The present disclosure will now be described more fully hereinafter with reference to the accompanying drawings illustrating various exemplary embodiments of the invention. The present disclosure may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

The term “substantially” means essentially conforming to the particular dimension, shape, or other feature that the term modifies, such that the component need not be exact. For example, “substantially cylindrical” means that the object resembles a cylinder, but may have one or more deviations from a true cylinder. The term “comprising” or “containing” when utilized, means “including, but not necessarily limited to”; it specifically indicates open-ended inclusion or membership in the so-described combination, group, series and the like. The terms “first”, “second”, “third” and other terms in the present disclosure are only used as textual symbols as the circumstances may require, but such a practice of ordination is not limited to using only these terms. It should be further noted that these terms can be used interchangeably.

It will be understood that the terms “and/or” and “at least one” include any and all combinations of one or more of the associated listed items. It will also be understood that, although the terms first, second, third etc. may be used herein to describe various elements, components, regions, parts and/or sections, these elements, components, regions, parts and/or sections should not be limited by these terms. These terms are only used to distinguish one element, component, region, part or section from another element, component, region, layer or section. Thus, a first element, component, region, part or section discussed below could be termed a second element, component, region, layer or section without departing from the teachings of the present disclosure.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

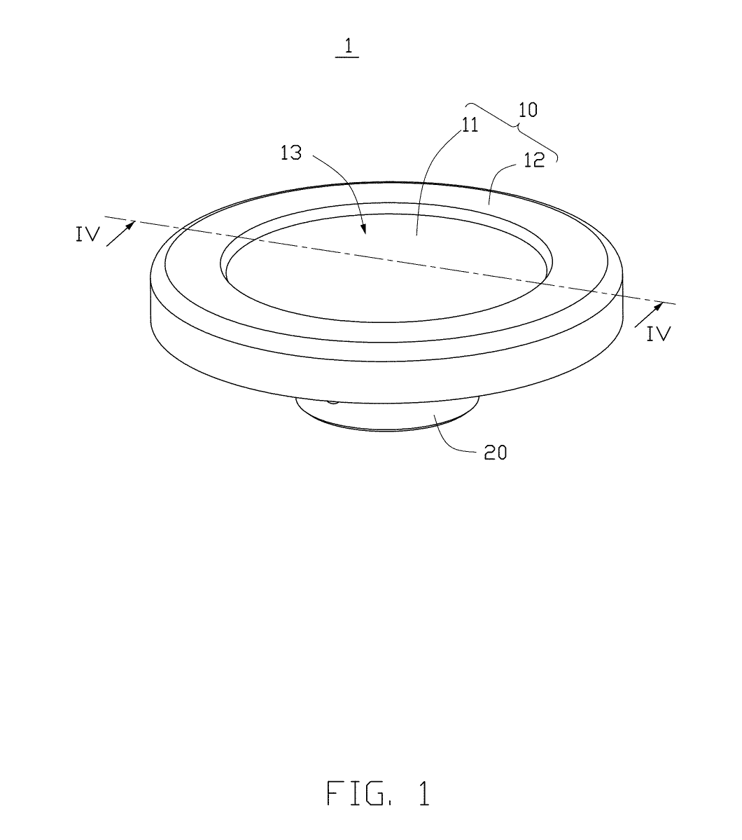

Referring to FIG. 1, a schematic illustration of a stethoscope head assembly is provided in accordance with an embodiment of the present disclosure. A stethoscope head assembly 1 comprises a diaphragm 10 and a main body 20 detachably coupled to the diaphragm 10. The diaphragm 10 comprises a membrane portion 11 and a sealing portion 12 extending from a periphery of the membrane portion 11. The membrane portion 11 may be integrally formed with the sealing portion 12, so the integral structure improves sound-gathering ability and makes it easy to assemble the diaphragm 10 with main body 20.

The membrane portion 11 and the sealing portion 12 cooperatively form a concave inducting space 13 as shown in FIG. 1, so environmental noise is isolated during auscultation.

The diaphragm 10 can be made of an elastic material, to facilitate comfortable use of the diaphragm 10. The elastic material may comprise a material with characteristics of thermal insulation and a Shore A hardness of substantially 30 to 80. The elastic material may also have a thermal deformation temperature of at least above 50 degrees, a high impact strength, and a high wear resistance. The elastic material may be silicon, thermoset rubber, thermoplastic rubber, or a combination thereof. Therefore, the characteristics of the material of the diaphragm 10 provide sufficient tension for the diaphragm 10 to be fixed on the main body 20, and to improve a sound gathering effect of the diaphragm 10 at a wide range of frequencies. It is also convenient to remove or replace the diaphragm 10 from the main body 20.

In the exemplary embodiment, the diaphragm 10 may be circular. In another exemplary embodiment, the diaphragm 10 may also be elliptical. The diaphragm 10 may be designed to have a suitable size and/or shape, to accommodate the main body 20.

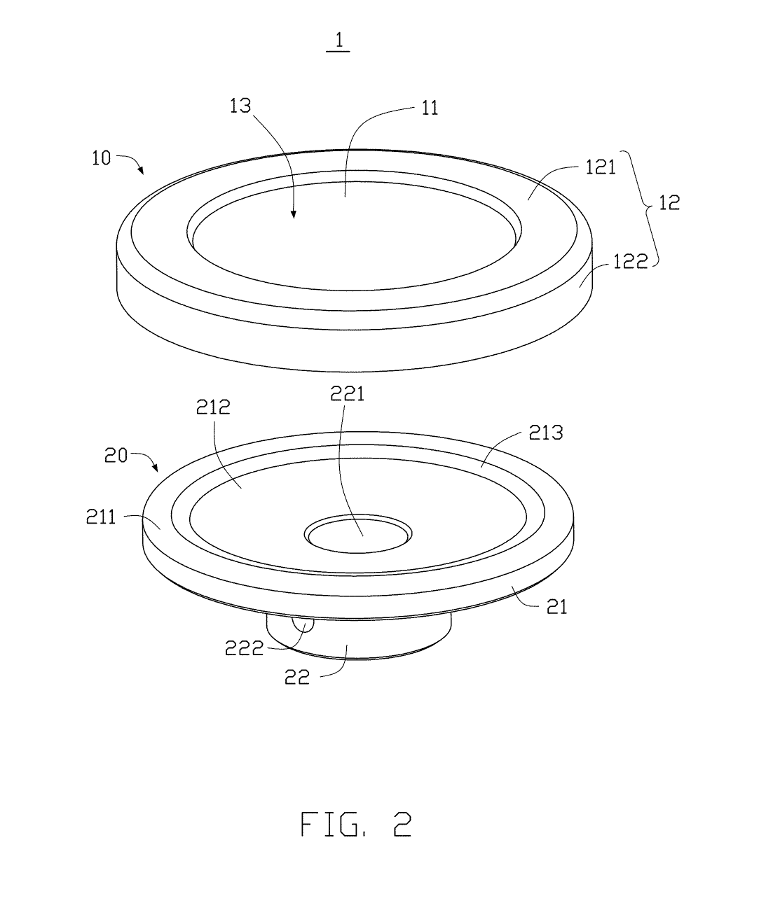

Referring to FIG. 2, an exploded view of the stethoscope head assembly is provided in accordance with an embodiment of the present disclosure. FIG. 2 shows the diaphragm 10 and the main body 20 are circular, and the membrane portion 11 of the diaphragm 10 is also substantially circular. The membrane portion 11 is configured to conduct vibration when the diaphragm 10 of the stethoscope head assembly 1 is being hold to against a patient. A thickness of the membrane portion 11 may be preferably in a range of substantially 0.05 mm to 1 mm. A diameter of the membrane portion 11 may be in a range of substantially 30 mm to 50 mm. A ring-shaped raised platform 121 extends from the membrane portion 11, and a connecting portion 122 extends downwardly from a periphery of the ring-shaped raised platform 121.

The main body 20 comprises a sound gathering member 21 and a sound conducting member 22 protruding downward from a center of the gathering member 21. The sound gathering member 21 may be integrally formed with the sound conducting member 22. An engaging portion 211 is arranged at a periphery of the sound gathering member 21. The sound gathering member 21 comprises a top surface 212 facing and directly contacting the diaphragm 10 when the diaphragm 10 is fixed to the main body 20. The main body 20 is substantially a funnel, and the main body 20 may be also made of an elastic material or a rigid material. The surface of the main body 20 may be partly covered by a layer of aluminum alloy. The sound conducting member 22 comprises a sound conducting hole 221 linked with a sound conducting tube 222. The top surface 212 of the sound gathering member 21 has a second matching element 213 adjacent to the engaging portion 211.

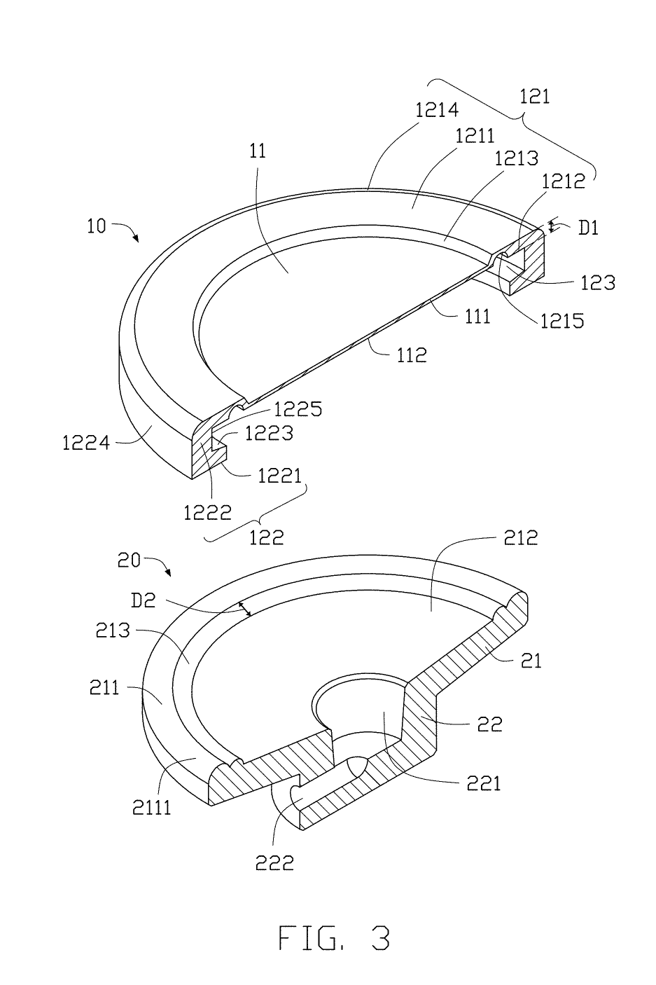

Referring to FIG. 3, an exploded cross-sectional view of the stethoscope head assembly is provided in accordance with an embodiment of the present disclosure. The sealing portion 12 comprises a ring-shaped raised platform 121 and a connecting portion 122 extending downwardly from a periphery of the ring-shaped raised platform 121. The connecting portion 122 may be vertically extending from the periphery of the ring-shaped raised platform 121. The ring-shaped raised platform 121 and the connecting portion 122 cooperatively form a ring-shaped receiving slot 123 for receiving the main body 20. When the main body 20 is received in the receiving slot 123, the membrane portion 11 may be secured in place by the sealing portion 12. The membrane portion 11 comprises a top surface 111 and a bottom surface 112. A thickness of the membrane portion 11 is less than a thickness D1 of the ring-shaped raised platform 121.

The ring-shaped raised platform 121 comprises a platform top surface 1211, a platform bottom surface 1212, a ring-shaped first side wall 1213, and a ring-shaped second side wall 1214. The ring-shaped first side wall 1213 protrudes inwardly from an inner periphery of the platform top surface 1211, and the ring-shaped second side wall 1214 protrudes outwardly from an outer periphery of the platform top surface 1211. A first matching element 1215 is arranged adjacent to the membrane portion 11 at the platform bottom surface 1212 of the raised platform 121. The ring-shaped first side wall 1213 of the raised platform 121 extends from the top surface 111 of the membrane portion 11. The ring-shaped first side wall 1213 of the raised platform 121 and the top surface 111 of the membrane portion 11 cooperatively form the concave inducting space 13 as shown in FIG. 1 and FIG. 2, so environmental noises are isolated during auscultation. An area of friction (not shown) or potential friction (not shown) between the body of the patient and the diaphragm 10 is small and reduces excess noise.

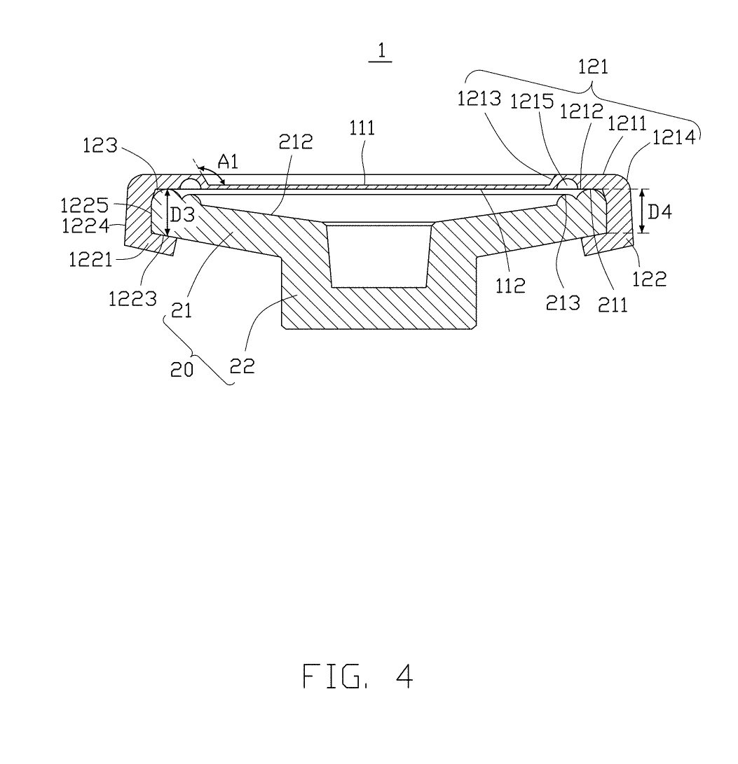

The ring-shaped first side wall 1213 is inclined. The ring-shaped first side wall 1213 protrudes away from a periphery of the top surface 111 of the membrane portion 11. The thickness of the ring-shaped first side wall 1213 is increased gradually in a direction away from the membrane portion 11, to avoid rupture of the diaphragm 10 during assembly. The ring-shaped first side wall 1213 with gradual thickness variation can provide elasticity for a user to change between a low or a high frequency mode of the stethoscope head assembly 1. The ring-shaped first side wall 1213 of the raised platform 121 and the top surface 111 of the membrane portion 11 form an angle A1 (shown in FIG. 4). The angle A1 may preferably fall within a range of substantially 120 degrees to 180 degrees.

In FIG. 3, an engaging portion 211 of the main body 20 can be received by the receiving slot 123 of the diaphragm 10. The engaging portion 211 comprises an engaging arc 2111, so the engaging portion 211 can be rotationally received in the receiving slot 123. The second matching element 213 is adjacent to the engaging portion 211 and is eccentric with the engaging portion 211. The second matching element 213 conforms in shape with the first matching element 1215 of the raised platform 121. A width D2 of the second matching element 213 is in a range of substantially 1 mm to 3 mm. An area on the diaphragm 10 projected by the top surface 212 is substantially consistent with an area on the diaphragm 10 circled by the second matching element 1215 of the raised platform 121.

The sound conducting hole 221 has a diameter of no larger than ½ of an outer diameter of the sound gathering member 21. The bottom of the sound conducting hole 221 is linked with the sound conducting tube 222. The sound conducting tube 222 has an exit on the lateral side of the sound conducting member 22, and the sound conducting tube 222 protrudes inward from the lateral side exit to the bottom of the sound conducting hole 221.

Referring to FIG. 4, a cross-sectional view of a relaxed state of the stethoscope head assembly is provided in accordance with an embodiment of the present disclosure. A cross-section of the connecting portion 122 is substantially L-shaped. The connecting portion 122 comprises a latching portion 1221 and a resisting portion 1222 coupled between the latching portion 1221 and the raised platform 121. The latching portion 1221 comprises a resisting surface 1223 substantially parallel with the platform bottom surface 1212 of the raised platform 121. The resisting portion 1222 comprises a first side surface 1224 and a second side surface 1225. The first side surface 1224 of the connecting portion 122 is adjacent to the ring-shaped second side wall 1214 of the raised platform 121, and a smooth transition is between the first side surface 1224 and the ring-shaped second side wall 1214. The resisting surface 1223, the second side surface 1225, and the platform bottom surface 1212 of the raised platform 121 cooperatively form an inner wall of the receiving slot 123. The inner wall of the receiving slot 123 may be configured as a concave surface, to facilitate the sealing formed between the main body 20 and the sealing portion 12. In the relaxed state, the second matching element 213 is not in contact with the second matching element 1215.

The cross-section of the sound gathering member 21 comprises the second matching element 213 and the engaging portion 211. The outer diameter of the sound gathering member 21 is at least equal to or greater than an outer diameter of the receiving slot 123. A thickness D3 of the engaging portion 211 of the sound gathering member 21 is substantially equal to a height D4 of the receiving slot 123.

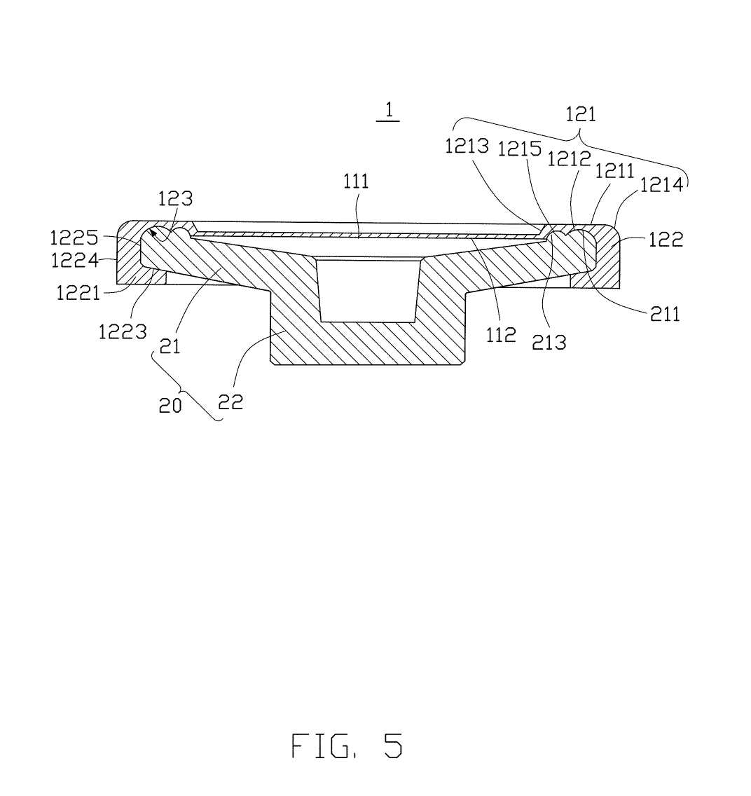

Referring to FIG. 5, a cross-sectional view of a compressed state of the stethoscope head assembly 1 is provided in accordance with an embodiment of the present disclosure. The stethoscope head assembly 1 in FIG. 4 is compressed by the user, and transformed into the stethoscope head assembly 1 in FIG. 5. In the compressed state, the second matching element 213 is in contact with the receiving slot 123, therefore the second matching element 213 is in contact with the first matching element 1215 and abuts against the first matching element 1215, and the space of the receiving slot 123 is occupied by the engaging portion 211. A slope of the top surface 212 in the relaxed state is steeper than the slope in the compressed state. A distance between the bottom of the sound conducting member 22 and the bottom surface 112 in the relaxed state is longer than the distance in the compressed state.

As shown in FIG. 4, the relaxed state of the stethoscope head assembly 1 represents a high frequency mode. The stethoscope head assembly 1 is transformed from the relaxed state to the compressed state by an external pressure, and the compressed state represents a low frequency mode. The external pressure may be applied by the user.

When the stethoscope head assembly 1 is in the high frequency mode, as illustrated by FIG. 4, the stethoscope head assembly 1 can be applied to the skin of the patient (not shown). The diaphragm 10 of the stethoscope head assembly 1 is in light contact with the skin of the patient, and the second matching element 213 of the main body 20 is not in contact with the first matching element 1215 of the raised platform 121. The bottom surface 112 of the membrane portion 11 is also more distant from the top surface 212 of the sound gathering member 21. The engaging portion 211 abuts against the resisting surface 1223 of the latching portion 1221 and the second side surface 1225 of the 1222 resisting portion. A resonating area (not shown) of the diaphragm 10 is thus larger, to enable the accentuation of high frequency sounds in the diaphragm 10. The high frequency sound created by the diaphragm 10 is gathered by the sound gathering member 21 and the sound conducting member 22, and then conveyed to the earpiece (not shown).

When the stethoscope head assembly 1 is in the low frequency mode, as illustrated by FIG. 5, an increased pressure to the sound gathering member 21 of the main body 20 can be applied by the user, resulting in additional force being applied to the skin of the patient. The diaphragm 10 is deformed until the first matching element 1215 fits the second matching element 213, and the diaphragm 10 of the stethoscope head assembly 1 is in higher-pressure contact with the skin of the patient. As a consequence of the increased pressure, the second matching element 213 of the main body 20 abuts against the first matching element 1215 of the raised platform 121. The engaging portion 211 abuts against the platform bottom surface 1212 of the raised platform 121 and the second side surface 1225 of the resisting portion 1222. Thereby, the resonating area (not shown) of the diaphragm 10 is smaller, to enable the accentuation of low frequency sounds in the diaphragm 10. The low frequency sound created by the diaphragm 10 is gathered by the sound gathering member 21, and then conveyed to the earpiece (not shown) by the sound conducting member 22.

The first matching element 1215 can be a protrusion when the second matching element 213 is a recess, and in the compressed state of the stethoscope head assembly 1, the first matching element 1215 abuts against an inner wall of the second matching element 213, and the first matching element 1215 is received by the second matching element 213. In another embodiment, the first matching element 1215 can be a recess when the second matching element 213 is a protrusion, and in the compressed state of the stethoscope head assembly 1, the second matching element 213 abuts against an inner wall of the first matching element 1215, and the second matching element 213 is received by the first matching element 1215, as shown in FIG. 5.

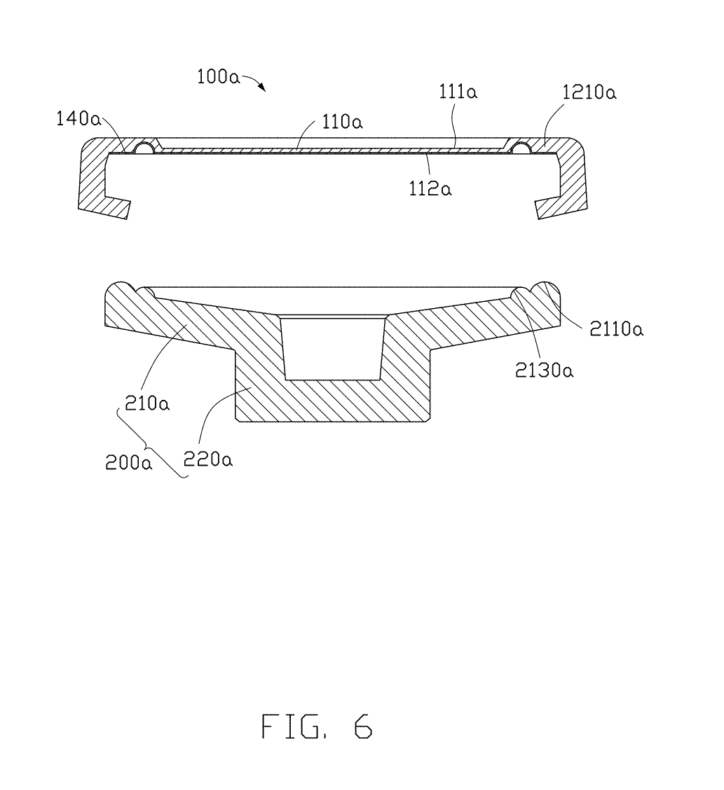

Referring to FIG. 6, a cross-sectional view of a stethoscope head assembly is provided in accordance with an embodiment of the present disclosure. A diaphragm 100a comprises a membrane portion 110a and a coating layer 140a. The membrane portion 110a further comprises a top surface 111a, a bottom surface 112a, and a ring-shaped raised platform 1210a. A main body 200a comprises a sound conducting member 220a and a sound gathering member 210a, and the gathering member comprises an engaging portion 2110a, and a protrusion 2130a adjacent to and eccentric with the engaging portion 2110a. The coating layer 140a enhances the sealing between the diaphragm 100a and the sound gathering member 200a, specifically the sealing between the ring-shaped raised platform 1210a, the protrusion 2130a, and the engaging portion 2110a. The coating layer 140a is disposed on the bottom surface 112a of the membrane portion 110a. The bottom surface 112a is fully covered by the coating layer 140a in FIG. 6. The coating layer 140a has greater elasticity than the membrane portion 110a, and is composed of elastic material that can be reversibly deformed when being pressed against the engaging portion 2110a and the protrusion 2130a of the sound gathering member 210a. The elastic material of the coating layer 140a can be silicon, soft tough paints, thermoplastic polyurethanes (TPU), thermoplastic rubbers (TPR), or the like. A thickness of the coating layer 140a is in a range of substantially 0.04 mm to 0.1 mm, preferably between 0.04 mm to 0.08 mm. The coating layer 140a may be constructed on the membrane portion 110a by spraying, injection molding, in-mold injecting, or the like. In the exemplary embodiment, a liquefied elastic material is sprayed onto the bottom surface 112a of the membrane portion 110a, follows by a solidification process to solidify the liquefied elastic material to form the coating layer 140a. The temperature of the solidification process is in a range of about 45° C. to 50° C.

The diaphragm 100a may further comprise a top layer (not shown). The top layer can be disposed on the top surface 111a of the membrane portion 110a. The top layer is composed of one or more elastic materials. The elastic material of the top layer can be silicon, soft tough paints, TPU, TPR, or the like. A thickness of the top layer is in a range of substantially 0.04 mm to 0.1 mm, preferably between 0.04 mm to 0.08 mm. The top layer may be constructed on the membrane portion 110a by spraying, injection molding, in-mold injecting, or the like.

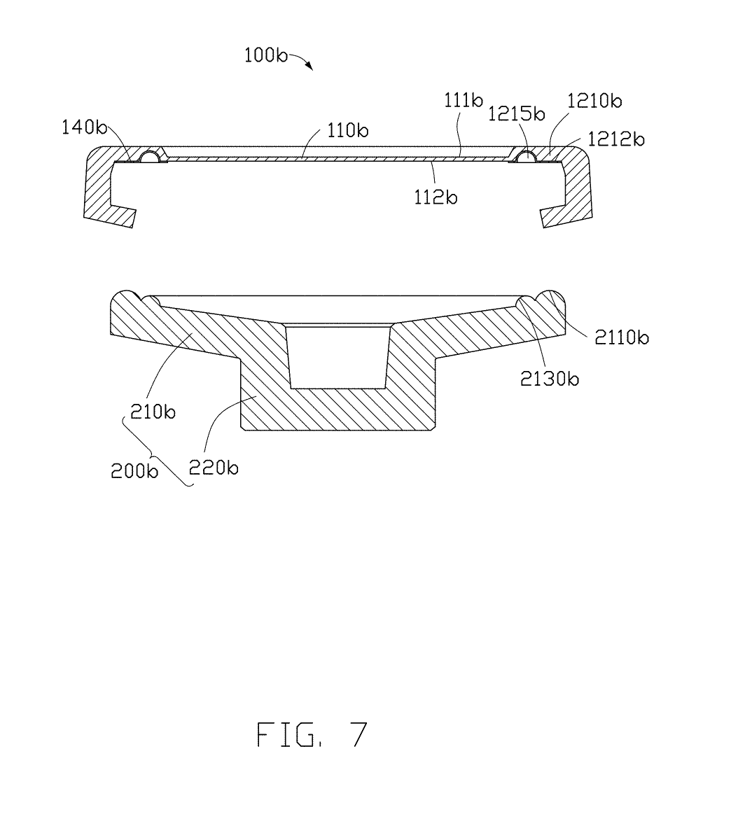

Referring to FIG. 7, a cross-sectional view of another stethoscope head assembly is provided in accordance with an embodiment of the present disclosure. A diaphragm 100b comprises a membrane portion 110b and a coating layer 140b. The membrane portion 110b further comprises a top surface 111b, a bottom surface 112b, and a ring-shaped raised platform 1210b. The ring-shaped raised platform comprises a platform bottom surface 1212b and a recess 1215b. A main body 200b comprises a sound conducting member 220b and a sound gathering member 210b, and the gathering member 210b comprises an engaging portion 2110b, and a protrusion 2130b adjacent to and eccentric with the engaging portion 2110b. The coating layer 140b enhances the sealing between the diaphragm 100b and the sound gathering member 200b, specifically the sealing between the ring-shaped raised platform 1210b, the protrusion 2130b, and the engaging portion 2110b. The coating layer 140b is disposed on the bottom surface 112b of the membrane portion 110b. The bottom surface 112b is partially covered by the coating layer 140b in FIG. 7. Specifically, the coating layer 140b is disposed on the platform bottom surface 1212b of the raised platform 1210b and the first matching element 1215b.

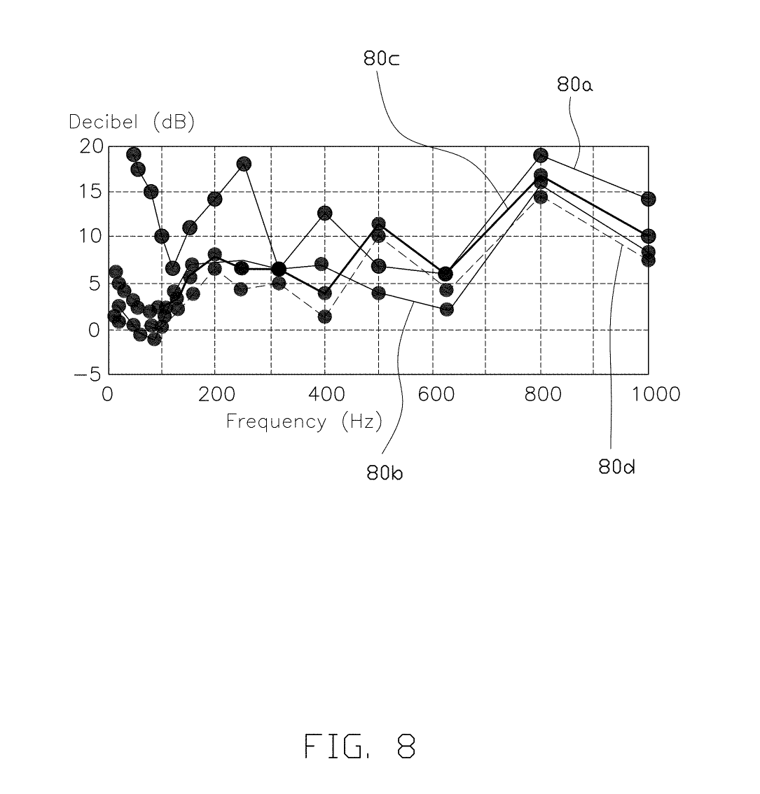

Referring to FIG. 8, results of gathering different frequencies of sounds by using stethoscope head assemblies with diaphragms of specific materials and main bodies of different diameters, in accordance with embodiments of the present disclosure. A stethoscope head assembly 80a comprises a PC diaphragm and a main body with an aluminum alloy layer on the surface of the main body, and a diameter of the main body is 28.5 mm. Another stethoscope head assembly 80b comprises a silicone diaphragm and a main body with an aluminum alloy layer on the surface of the main body, and a diameter of the main body is 28.5 mm. Table 1 shows results of sound intensities in different frequencies of sounds gathered by the stethoscope head assembly 80a and 80b. FIG. 8 shows a decibel (dB) value of the sound intensity under different frequencies bands using the stethoscope head assembly 80a and 80b.

TABLE 1

| | Acoustic Attenuation | Acoustic Attenuation |
|---|---|---|
| | Value in dB | Value in dB |
| | (Stethoscope Head | (Stethoscope Head |
| Frequency (Hz) | Assembly 80a) | Assembly 80b) |
| 20-100 | 19.4 | 2.6 |
| 20-200 | 17.1 | 3.5 |
| 200-600 | 10.6 | 5.5 |
| 100-600 | 10.3 | 5.0 |
| 20-800 | 15.0 | 4.7 |
| 20-1000 | 15.0 | 4.9 |

Referring to Table 1, the stethoscope head assembly 80a has an acoustic attenuation value of 19.4 dB in 20-100 Hz, and 10.6 dB in 200-600 Hz. The stethoscope head assembly 80b has an acoustic attenuation value of 2.6 dB in 20-100 Hz, and 5.5 dB in 200-600 Hz. Relative error between the stethoscope head assembly 80a and 80b is in a range of 5.1 dB to 15.8 dB. Therefore, the stethoscope head assembly 80b is better for diagnosing the patient with pathological conditions at different frequencies, because the stethoscope head assembly 80b shows lower acoustic attenuation value than the stethoscope head assembly 80a.

Diaphragms of different diameters are also tested. In FIG. 8, a stethoscope head assembly 80c and 80d are provided in accordance with embodiments of the present disclosure. The stethoscope head assembly 80c comprises a PC diaphragm and a main body with an aluminum alloy layer on the surface of the main body, and a diameter of the main body is 43.5 mm. Another stethoscope head assembly 80d comprises a silicone diaphragm and a main body with an aluminum alloy layer on the surface of the main body, and a diameter of the main body is 43.5 mm. Table 2 shows results of sound intensities in different frequencies of sounds gathered by the stethoscope head assembly 80c and 80d.

TABLE 2

| | Acoustic Attenuation | Acoustic Attenuation |
|---|---|---|
| | Value in dB | Value in dB |
| Frequency | (Stethoscope Head | (Stethoscope Head |
| (Hz) | Assembly 80c) | Assembly 80d) |
| 20-100 | 3.7 | 1.0 |
| 20-200 | 4.2 | 1.9 |
| 200-600 | 7.1 | 5.7 |
| 100-600 | 5.9 | 4.7 |
| 20-800 | 5.7 | 3.7 |
| 20-1000 | 6.0 | 4.0 |

Referring to Table 2, the relative error between a stethoscope head assembly 80c and 80d is in a range of 1.2 dB to 2.7 dB. Table 2 shows that the stethoscope head assembly 80d is better for diagnosing the patient with pathological conditions at different frequencies, because the stethoscope head assembly 80d shows lower acoustic attenuation value in Table 2 and FIG. 8.

Table 1 and Table 2 show that the acoustic attenuation values of the stethoscope head assemblies are influenced by the material and the diameter of the main body. When 28.5-mm diameter main body is used, the stethoscope head assembly 80b has the acoustic attenuation value of 2.6 dB in 20-100 Hz. When 43.5-mm-diameter main body is used, the stethoscope head assembly 80d has an acoustic attenuation value of 1.0 dB in 20-100 Hz. Therefore, within the same frequency range, the larger the diameter of the main body, the smaller acoustic attenuation value it would be for the stethoscope head assemblies.

TABLE 3

| Sound intensity | Acoustic | Acoustic |
|---|---|---|
| (dB) | Energy Ratio | Pressure Ratio |
| 100 | 10000000000 | 100000 |
| 90 | 1000000000 | 31623 |
| 80 | 100000000 | 1000 |
| 70 | 10000000 | 3162 |
| 60 | 1000000 | 1000 |
| 50 | 100000 | 316.2 |
| 40 | 10000 | 100 |
| 30 | 1000 | 31.62 |
| 20 | 100 | 10 |
| 10 | 10 | 3.162 |
| 6 | 3.981 | 1.995(−2) |
| 3 | 1.995(−2) | 1.413 |
| 1 | 1.259 | 1.122 |
| 0 | 1 | 1 |

Table 3 shows that a relationship between an acoustic pressure ratio and an acoustic energy ratio. According to Table 1, the difference of the acoustic attenuation value between the stethoscope head assembly 80a and 80b in 20-1000 Hz is 10.1 dB. Table 3 indicates the acoustic energy ratio is 3.162 when the acoustic pressure ratio is 10, therefore the stethoscope head assembly 80b has improved by 3.162 times relative to the stethoscope head assembly 80a. According to Table 2, the difference of the acoustic attenuation value between the stethoscope head assembly 80c and 80d in 20-1000 Hz is 2 dB. Table 3 indicates the acoustic energy ratio is 1.413 when the acoustic pressure ratio is 3, and 1.122 when the acoustic pressure ratio is 1. Therefore, when the acoustic pressure ratio is 2, it can be calculated that the acoustic energy ratio is 1.3 by interpolation. Thus, the stethoscope head assembly 80d has improved by 1.3 times relative to the stethoscope head assembly 80c.

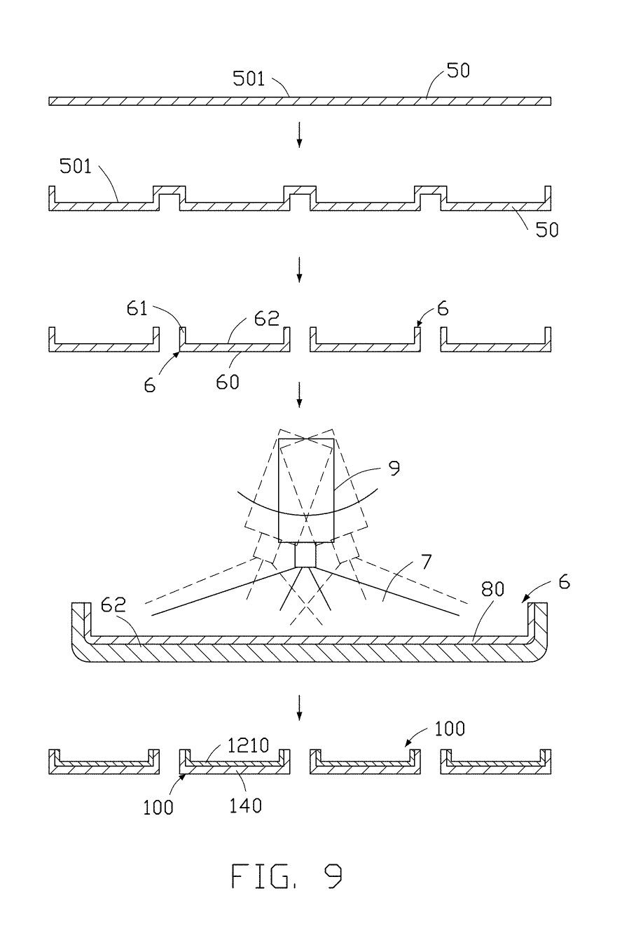

Referring to FIG. 9, a manufacturing method of the diaphragm is provided in accordance with an embodiment of the present disclosure. Firstly, provide a flat film 50 which comprises a first surface 501. Secondly, perform a preforming and stamping step to the flat film 50 for forming a plurality of semi-finished membranes 6, each of the semi-finished membranes comprises a flat membrane portion 60 and a pair of the connecting portions 61 extending from periphery of the membrane portion 60 towards a first surface 62 of the membrane portion 60. Thirdly, spray the liquefied elastic material 7 onto the first surface 62 of the semi-finished membrane 6. Finally, perform a solidification step to the liquefied elastic material 7 to form an intermediate coating layer 80. A plurality of diaphragms 100 including the coating layer 140 and the connecting portions 1210 are thus produced.

In the exemplary embodiment, a four-axis dispenser 9 is adopted for rotatably spraying the liquefied elastic material onto both the first surface 62 of the membrane portion 60 and the connecting portions 61. The four-axis dispenser 9 facilitates the manufacturing process and reduces defect rate.

In the exemplary embodiment, the diaphragm may also be manufactured by three-dimensional printing (3D printing) methods, such as sterolithography (SLA), digital light processing (DLP), fused deposition modeling (FDM), selective laser sintering (SLD), selective laser melting (SLM), electronic beam melting (EBM), or laminated object manufacturing (LQM).

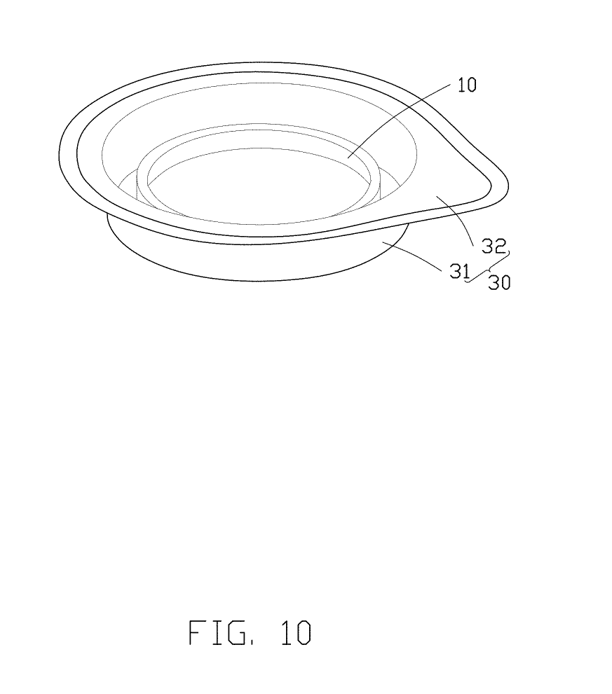

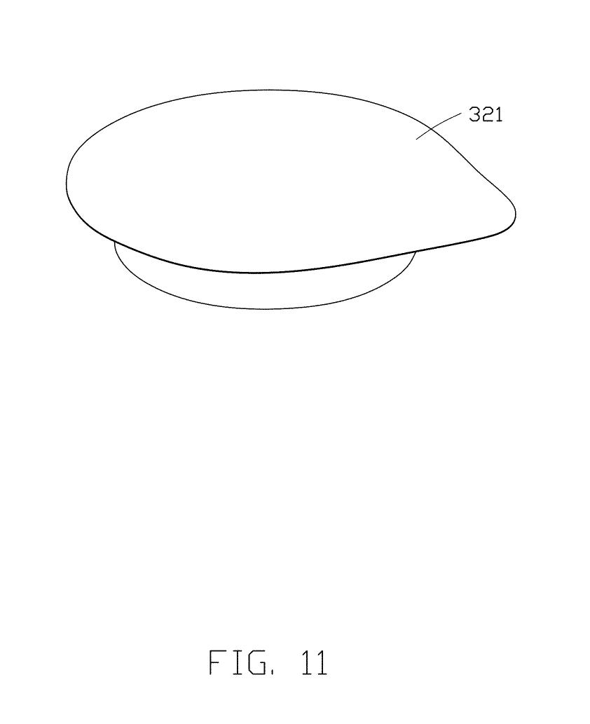

In the exemplary embodiment, the diaphragm may be designed as a single-use, disposable product for hygiene concerns. Referring to FIG. 10 and FIG. 11, disposable capsules for the diaphragm is provided in accordance with an embodiment of the present disclosure. The diaphragm 10 for replacement may be contained in a disposable capsule 30 as shown in FIG. 10. The disposable capsule 30 may comprise a blister 31, a sealing film 32 and a diaphragm 10. The blister 31 may be composed of polymer materials comprising PS, PET, PC, or materials such as metal or cardboard. The color of blister depends on the material being used. The sealing film may be composed of paper, PS, PET, PC or composites such as “Easy Peel Film™” or “Tavik paper” 321, as shown in FIG. 11. The diaphragm 10 comprises a sealing portion 12 for bonding with the engaging portion 211 of the stethoscope 20. The sealing portion 12 may be made of silicon or other flexible material. A method for sealing the blister 31 and the sealing film is hot-press gluing or ultrasonic welding. In another exemplary embodiment, a groove line is die-punched on the blister. The sealing film 32 may be torn easily by bending and breaking a part of blister 31 along the groove line as shown in FIG. 10. The whole disposable capsule can be further sterilized.

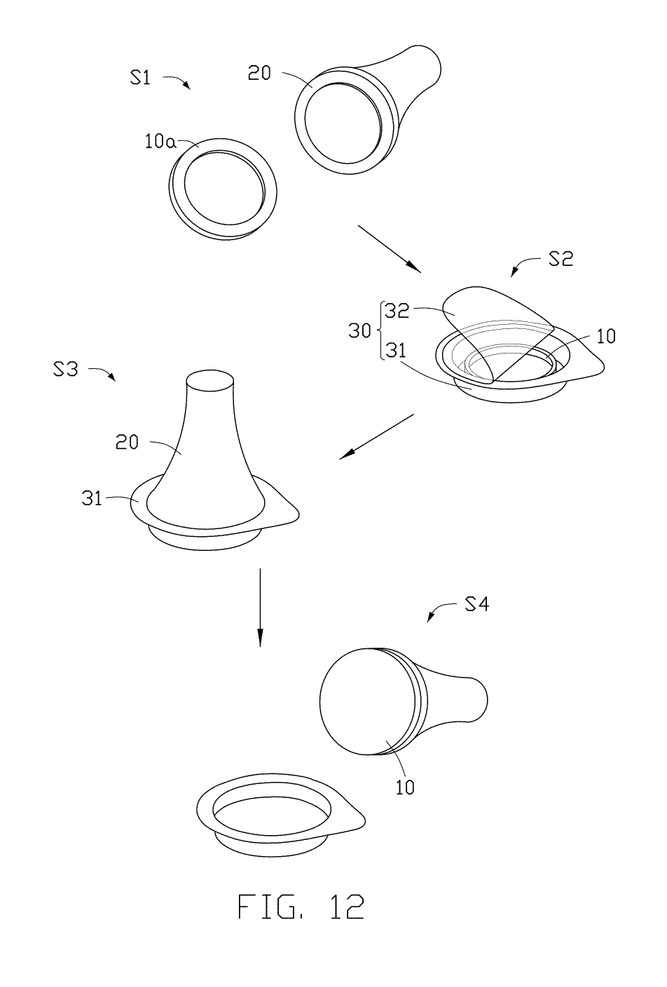

Referring to FIG. 12, a method for replacing the diaphragm of a stethoscope using the disposable capsule is provided in accordance with an embodiment of the present disclosure. The method for replacing the diaphragm comprises the following steps: S1: Remove a used diaphragm 10c from a main body 20 of a stethoscope head assembly. S2 Tear the sealing film 32 from the disposable capsule 30. S3: Push the main body 20 of the stethoscope into the diaphragm 10 against the blister 31 when the diaphragm is still in the disposable capsule 30, so that the sealing portion of the diaphragm 10 is firmly attached with the main body 20 of the stethoscope. S4: Pull the main body 20 out of the disposable capsule 30. The diaphragm 10 of the stethoscope is replaced without being touched.

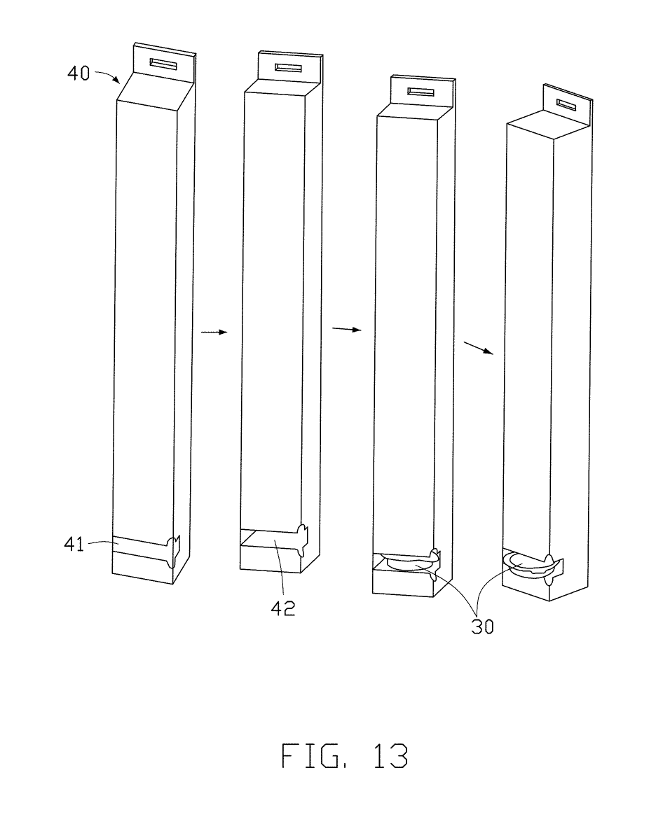

Referring to FIG. 13, a package for the disposable capsules is provided in accordance with an embodiment of the present disclosure. If the stethoscope head assembly needs to be frequently changed, a package 40 can be provided to facilitate retrieval of the disposable capsules 30. As shown in FIG. 13, the package 40 comprises a tearing portion 41 and a plurality of disposable capsules 30. The tearing portion 41 is configured to form an opening 42 after being torn off for retrieving the disposable capsule 30 out of the package 40. The plurality of the disposable capsules 30 is stacked inside the package 40 so that the next disposable capsule 30 will slide down to the place of the opening 42 when one disposable capsule 30 is taken away from the opening 42. In the exemplary embodiment, the package may further comprise a fixing portion to fix the package in a desired place such as on a table or on a wall. The fixing portion may be a hook or an adhesive.

Previous descriptions are only embodiments of the present disclosure and are not intended to limit the scope of the present disclosure. Many variations and modifications according to the claims and specification of the disclosure are still within the scope of the claimed disclosure. In addition, each of the embodiments and claims does not have to achieve all the advantages or characteristics disclosed. Moreover, the abstract and the title only serve to facilitate searching patent documents and are not intended in any way to limit the scope of the claimed disclosure.

What is claimed is:

1. A diaphragm for a stethoscope head assembly, comprising:
a membrane portion; and
a sealing portion, extending from a periphery of the membrane portion and comprising a ring-shaped raised platform and a connecting portion extending downwardly from a periphery of the ring-shaped raised platform, wherein the connecting portion and the ring-shaped raised platform cooperatively form a ring-shaped receiving slot.

2. The diaphragm according to claim 1, wherein the diaphragm is made of an elastic material with a Shore A hardness of 30 to 80, and the elastic material comprises silicon, thermoset rubber, thermoplastic rubber, or a combination thereof.

3. The diaphragm according to claim 1, wherein a thickness of the membrane portion is less than a thickness of the ring-shaped raised platform of the sealing portion.

4. The diaphragm according to claim 3, wherein the thickness of the membrane portion falls within a range of 0.05 mm to 1 mm, and a diameter of the membrane portion falls within a range of 30 mm to 50 mm.

5. The diaphragm according to claim 1, wherein the ring-shaped raised platform comprises a platform top surface, a platform bottom surface, a ring-shaped first side wall protruding inwardly from a periphery of the platform top surface, a ring-shaped second side wall protruding outwardly from another periphery of the platform top surface, and
the connecting portion comprises a latching portion and a resisting portion coupled between the latching portion and the ring-shaped raised platform.

6. The diaphragm according to claim 5, wherein the ring-shaped first side wall of the ring-shaped raised platform and a top surface of the membrane portion form an angle, and the angle falls within a range of 120 degrees to 180 degrees.

7. The diaphragm according to claim 1, wherein the sealing portion further comprises a first matching element arranged adjacent to the membrane portion and at the platform bottom surface of the ring-shaped raised platform.

8. The diaphragm according to claim 7, wherein the first matching element is a recess.

9. The diaphragm according to claim 7, wherein the first matching element is a protrusion.

10. The diaphragm according to claim 1, further comprising a coating layer disposed on a bottom surface of the membrane portion and a platform bottom surface of the ring-shaped raised platform.

11. The diaphragm according to claim 10, wherein the coating layer is made of silicon, soft touch paint, thermoplastic polyurethane, thermoplastic rubber, or a combination thereof.

12. The diaphragm according to claim 1, wherein the diaphragm is manufactured by 3D printing.

13. A stethoscope head assembly, comprising:
a diaphragm comprising:
a membrane portion;
a sealing portion, extending from a periphery of the membrane portion and comprising
a ring-shaped raised platform;
a first matching element, arranged adjacent to the membrane portion at a bottom surface of the ring-shaped raised platform; and
a connecting portion extending downwardly from a periphery of the ring-shaped raised platform;
a main body, detachably coupled to the diaphragm and comprising
a sound gathering member;
wherein the sound gathering member comprises a second matching element aligned to conform in shape with the first matching element of the ring-shaped raised platform at a top surface of the sound gathering member, and the connecting portion and the ring-shaped raised platform of the diaphragm cooperatively form a ring-shaped receiving slot for receiving the sound gathering member of the main body.

14. The stethoscope head assembly according to claim 13, wherein the diaphragm is made of an elastic material with a Shore A hardness of 30 to 80, and the elastic material comprises silicon, thermoset rubber, thermoplastic rubber, or a combination thereof.

15. The stethoscope head assembly according to claim 13, wherein a thickness of the membrane portion is less than a thickness of the ring-shaped raised platform of the sealing portion.

16. The stethoscope head assembly according to claim 15, wherein the thickness of the membrane portion falls within a range of 0.05 mm to 1 mm, and a diameter of the membrane portion falls within a range of 30 mm to 50 mm.

17. The stethoscope head assembly according to claim 13, wherein the ring-shaped raised platform comprises a platform top surface, a platform bottom surface, a ring-shaped first side wall protruding inwardly from a periphery of the platform top surface, a ring-shaped second side wall protruding outwardly from another periphery of the platform top surface, and
the connecting portion comprises a latching portion and a resisting portion coupled between the latching portion and the ring-shaped raised platform.

18. The stethoscope head assembly according to claim 17, wherein the ring-shaped first side wall of the ring-shaped raised platform and a top surface of the membrane portion form an angle, the angle falls within a range of 120 degrees to 180 degrees.

19. The stethoscope head assembly according to claim 13, wherein a width of the second matching element falls within a range of 1 mm to 3 mm.

20. The stethoscope head assembly according to claim 13, further comprising a relaxed state where the first matching element is not in contact with the second matching element, and a compressed state where the first matching element is in contact with the second matching element and abuts against the second matching element, wherein the stethoscope head assembly is transformed from the relaxed state to the compressed state by an external pressure.

21. The stethoscope head assembly according to claim 13, wherein the first matching element is a recess, and the second matching element is a protrusion.

22. The stethoscope head assembly according to claim 13, wherein the first matching element is a protrusion, and the second matching element is a recess.

23. The stethoscope head assembly according to claim 13, wherein an outer diameter of the ring-shaped receiving slot is equal to an outer diameter of the sound gathering member.

24. The stethoscope head assembly according to claim 13, further comprising an engaging portion of the sound gathering member arranged on a periphery of the sound gathering member, wherein a thickness of the engaging portion of the sound gathering member is equal to a height of the ring-shaped receiving slot.

25. The stethoscope head assembly according to claim 13, further comprising a coating layer disposed on a bottom surface of the membrane portion and a platform bottom surface of the ring-shaped raised platform.

26. The stethoscope head assembly according to claim 25, wherein the coating layer comprises silicon, soft touch paint, thermoplastic polyurethane, thermoplastic rubber, or a combination thereof.

27. The stethoscope head assembly according to claim 13, wherein the diaphragm is manufactured by 3D printing.
